(19) United States
(12) Patent Application Publication
(10) Pub. No.: US 2009/0035724 A1
(43) Pub. Date: Feb. 5, 2009

(54) Site Specific Intra-Oral Application Apparatus

(76) Inventors: Andrew Noel Quick, Dunedin (NZ); Dorothy Helen Boyd, Dunedin (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/908,711

(22) PCT Filed: Mar. 14, 2006
(86) PCT No.: PCT/NZ2006/000046

§ 371 Date: Jun. 9, 2008

(30) Foreign Application Priority Data
Mar. 14, 2005 (NZ) 538805

(51) Int. Cl. A61C 17/00; A61C 19/06; A61B 19/00

(52) U.S. Cl. 433/215; 433/82; 206/63.5

(57) ABSTRACT

An apparatus (7) for localised application of a substance to a site within the oral cavity has a reservoir (9) for receipt of the substance to be applied, and a mounting arrangement (11) a for mounting the apparatus within the oral cavity. The mounting arrangement (11) is configured to place the reservoir (9) in contact with the site to which the substance is to be applied to substantially form a seal between the reservoir and the site to which the substance is to be applied.

### FIELD OF THE INVENTION

The present invention relates to an apparatus suitable for localised application of a substance to a site within the oral cavity. More particularly, although not exclusively, the apparatus is suitable for localised application of a substance to the surface of a tooth.

### BACKGROUND TO THE INVENTION

Over time, damage and decay to the surfaces of teeth occurs as a result of plaque build up or poor oral hygiene or dental care. As well as possibly causing long term damage, this decay can be aesthetically unappealing. Regular use of toothpaste and/or mouth washes which contain fluoride or other active ingredients goes some way towards minimising plaque build up, thereby reducing long term decay and damage. However, the beneficial effects from the toothpaste or mouth washes are only relatively short term, as the substances are generally diluted in the saliva or removed from the tooth surfaces when liquid or food is consumed.

During orthodontic treatment using orthodontic appliances such as braces, enamel demineralisation of the teeth has been found to occur, especially in the cervical or neck areas of the teeth (the region around the crown and root interface of the tooth). This can result in discolouration of the teeth, as well as possible long term damage.

There are a number of apparatuses in existence that are designed for release of substances into the oral cavity. Such apparatuses are disclosed in U.S. Pat. No. 5,049,077; U.S. Pat. No. 5,137,449; and U.S. Pat. No. 5,074,786 for example. These apparatuses are designed for delivery of substances generally throughout the mouth, via the saliva. A disadvantage of each of these apparatuses is that the substance may be expended in a reasonably short period of time, as the substance will be readily dispersed by the salvia. Therefore, the apparatuses will likely require regular refilling with substance. Additionally, these apparatuses are wasteful of substance, especially if only specific areas of the mouth (such as specific areas of one or more teeth) require the substance to be applied.

U.S. Pat. No. 4,892,483 describes a retaining apparatus for holding a substance. This apparatus has a complex hinge and pin arrangement, and while the substance may be in relatively close contact with the tooth surface, the configuration of the substance holding portion of the apparatus is such that the substance is also dispersed throughout the oral cavity in the saliva. Again, this apparatus is relatively wasteful of substance, especially if only specific areas of the mouth (such as specific areas of one or more teeth) require the substance to be applied.

WO 01/68038 and US 2005/087171 describe the use of a matrix with an active ingredient which is held under an orthodontic arch wire for example. Again the active ingredient will be exposed for distribution in the salvia, and further the matrix will be prone to dislodging from under the wire.

US 2005/0196727; U.S. Pat. No. 6,997,706; and WO 2005/087171 describe a bracket that is adhered to the surface of a tooth. The bracket has a cavity that faces away from the tooth surface and holds a tablet for dispersion in the saliva.

Other dental devices are described in U.S. Pat. No. 4,681,544; WO 02/24100; U.S. Pat. No. 6,197,331; U.S. Pat. No. 6,685,928; U.S. Pat. No. 6,726,898; US 2004/0157187; and WO 04/041103.

It is an object of preferred embodiments of the present invention to provide an apparatus for localised application of a substance to a site within the oral cavity, and/or an associated method, which addresses at least one of the disadvantages outlined above or which at least provides the public with a useful choice.

### SUMMARY OF THE INVENTION

In accordance with a first aspect of the present invention, there is provided an apparatus for localised application of a substance to a site within the oral cavity, comprising a reservoir for receipt of the substance to be applied, and a mounting arrangement for mounting the apparatus within the oral cavity, with the mounting arrangement configured to place the reservoir in contact with the site to which the substance is to be applied to substantially form a seal between the reservoir and the site to which the substance is to be applied.

The term ‘comprising’ as used in this specification and claims means ‘consisting at least in part of’, that is to say when interpreting statements in this specification and claims which include that term, the features prefaced by that term in each statement all need to be present but other features can also be present. Related terms such as ‘comprise’ and ‘comprised’ are to be interpreted in a similar manner.

The mounting arrangement may be substantially rigid, or may be configured to bias the reservoir into contact with the site to which the substance is to be applied. In the embodiment in which the mounting arrangement is configured to bias the reservoir, the mounting arrangement may be resilient or may comprise a spring portion, such that once the apparatus is mounted in the oral cavity, the reservoir may be pulled away from the site against the bias to enable the reservoir to be refilled.

The mounting arrangement may be configured for attachment to an orthodontic appliance. In one embodiment, the mounting arrangement is configured for attachment to an orthodontic bracket which may be adhered to a tooth. In another embodiment, the mounting arrangement may be configured for attachment to an orthodontic arch wire.

Alternatively, the mounting arrangement may comprise a portion that is configured for attachment to a natural part of the oral cavity. The mounting arrangement may be configured for attachment to a surface of a tooth.

The reservoir may be pivotable relative to the mounting arrangement, to enhance the contact of the reservoir with the site in the oral cavity to which the substance is to be applied.

At least a perimeter of the reservoir may be relatively flexible, to enhance contact between the reservoir and the site in the oral cavity. The perimeter of the reservoir may have greater flexibility than at least a substantial part of the remainder of the reservoir, to enable the substantial part of the remainder to substantially retain its shape for containing the substance to be applied.

The apparatus is preferably configured to apply the substance to a surface of a tooth in the oral cavity. The apparatus may be configured to apply the substance to a neck region of a tooth in the oral cavity. Advantageously, the apparatus is configured to apply the substance to a neck region of a buccal surface of a tooth in the oral cavity.

Alternatively, the apparatus could be configured to apply a substance to an alternative site in the oral cavity.

The apparatus may comprise more than one reservoir, such that the apparatus is capable of concurrently locally applying one or more substances to more than one site in the oral cavity.

In accordance with a second aspect of the present invention, there is provided a kit comprising a plurality of apparatuses as outlined in relation to the first aspect above.

Suitably, the kit comprises six of said apparatuses.

The kit advantageously comprises at least one substance for application by the apparatuses.

In accordance with a third aspect of the present invention, there is provided a method of locally applying a substance to a site within an oral cavity, comprising mounting an apparatus as outlined in relation to the first aspect above in the oral cavity; and applying a substance from the reservoir to the site in the oral cavity.

The apparatus may have been pre-filled with the substance. Alternatively, the method may comprise inserting the substance into the reservoir after mounting the apparatus in the oral cavity.

The method may comprise, once a substance has been expended or following a predetermined time delay, moving the reservoir out of contact with the site, inserting more substance or a different substance in the reservoir, and bringing the reservoir back into contact with the site.

The method may be used to cosmetically enhance a user's teeth.

The invention consists in the foregoing and also envisages constructions of which the following gives examples only.

### BRIEF DESCRIPTION OF THE DRAWINGS

Preferred embodiments of the invention will be described with reference to the accompanying drawings in which:

FIG. 1 is a perspective view of three top teeth in the oral cavity of a human, showing a first preferred embodiment apparatus in contact with one of the teeth;

FIG. 2 is a sectional view along line 2-2 of FIG. 1;

FIG. 3 is a perspective view of an upper tooth with a second preferred embodiment apparatus in contact with the tooth;

FIG. 4 is a sectional view along line 4-4 of FIG. 3;

FIG. 5 is a perspective view of an upper tooth with a third preferred embodiment apparatus in contact with tooth; and

FIG. 6 is a perspective view of an upper tooth with a fourth preferred embodiment apparatus in contact with the tooth.

### DETAILED DESCRIPTION OF PREFERRED EMBODIMENTS

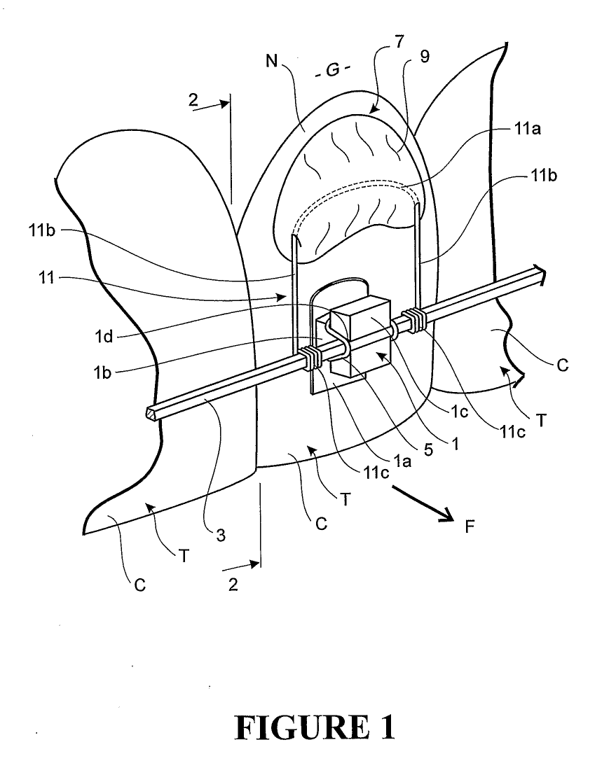

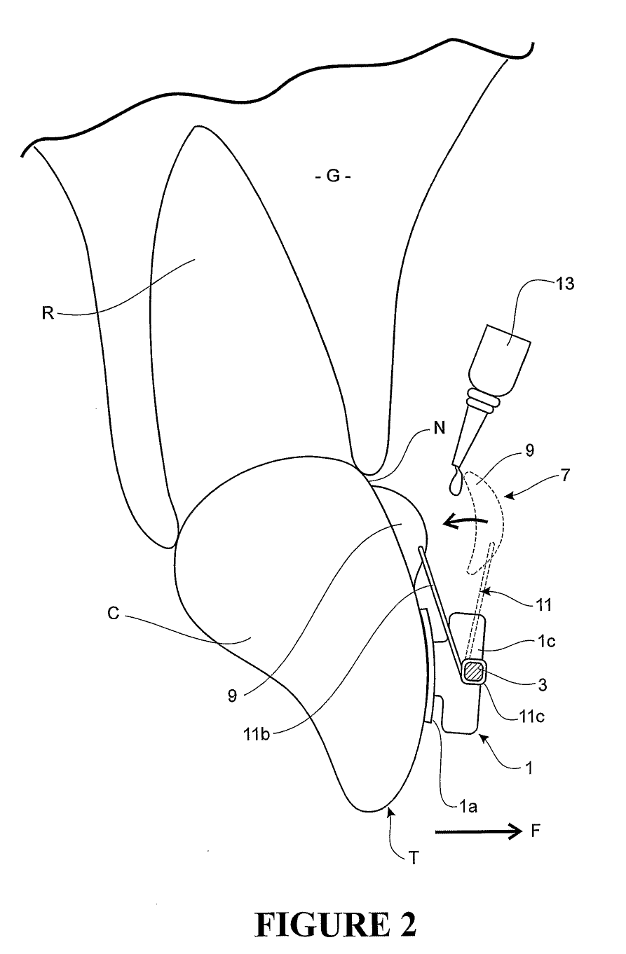

With reference to FIG. 1, three top teeth of an oral cavity are shown, with each tooth indicated by T. The teeth T extend from the gum G in the normal manner so that their crowns C are exposed. FIG. 1 shows the outer or buccal surfaces of the teeth T, i.e. the portions of the teeth which face the lip or the cheek. The direction of the front of the mouth is generally indicated by arrow F in the figures. FIG. 2 shows the neck area N which is the region around the crown C and root R interface of the tooth.

Reverting to FIG. 1, adhered to at least one of the teeth, and generally to a number of the teeth (although only one is shown) is an orthodontic bracket referenced by reference numeral 1. The bracket 1 will generally be of the type used to hold a braces arch wire 3. Each orthodontic bracket has a base portion 1a which is adhered to the tooth using a suitable adhesive, and has a neck 1b, and a holding or body portion 1c including a channel 1d for receipt of part of the orthodontic arch wire 3.

A band or retainer 5 extends around a portion of the bracket 1 to maintain the wire in position in the channel 1d. While only a single bracket is shown, it will be appreciated that similar brackets will generally be adhered to other teeth, as is standard for orthodontic procedures such as tooth straightening.

FIGS. 1 and 2 show a first preferred embodiment apparatus indicated generally by reference numeral 7 which is suitable for localised application of a substance to a site within the oral cavity. The first preferred embodiment apparatus comprises a reservoir 9 having an interior region for receipt of a substance to be applied to the tooth surface, as well as a mounting arrangement 11 for mounting the apparatus in a desired position within the oral cavity. The mounting arrangement 11 is configured to bring the reservoir into contact with the surface of the site in the oral cavity to which the substance is to be applied, in this embodiment to the surface of the tooth.

In the embodiment shown, the mounting arrangement 11 comprises one or more portions 11a (and preferably one portion 11a) which engage the reservoir, one or more arm portions 11b (and preferably two arm portions 11b), and one or more connecting portions 11c (and preferably two connecting portions 11c) which connect the apparatus to another orthodontic appliance, in this embodiment to the arch wire 3. In the particular embodiment shown, the mounting arrangement 11 exhibits some similarity to an anterior torquing spring, such as that marketed by Rocky Mountain Orthodontics of Denver, Colo., under the name WARREN SPRING. Such a spring is shown in U.S. Pat. No. 3,975,823, and the contents of that specification are incorporated herein by way of reference.

However, in this application, the mounting arrangement 11 will be provided with sufficient biasing force to maintain the reservoir in contact with the surface of the tooth to substantially form a seal about the reservoir's perimeter, but not sufficient biasing force to apply any significant torquing action to the tooth.

For strength and reliability, the apparatus will preferably be configured so that the arm portions 11b and connecting portions 11c straddle the bracket 1.

The portion(s) 11a which engage the reservoir could be adhered to the reservoir, or could be moulded into the reservoir for example. At least the part of the reservoir which contacts the surface of the tooth in use is suitably relatively flexible. In some embodiments, the entire reservoir may be relatively flexible. In alternative embodiments, part of the reservoir may be relatively rigid to enable it to hold sufficient shape to contain the substance, with the perimeter region of the reservoir which contacts the surface of the tooth in use being relatively flexible. To achieve that, the reservoir could be made from two different materials, with a more flexible material being used for the perimeter of the reservoir and a less flexible material being used for at least a major part of the remainder of the reservoir. The two parts of the reservoir could be co-moulded from the different materials for example. A suitable material for the perimeter is latex, flexible plastic, or biocompatible silicone polymer for example, and a suitable material for the remainder is a plastic, acrylic, or rubber-like material for example.

By providing a reservoir which is relatively flexible, or which at least has a perimeter which is relatively flexible, a good contact surface between the reservoir and the tooth will be achieved, to substantially form a seal there between and reduce any effect of saliva on the substance to be applied to the surface of the tooth. The reservoir may be made from a suitable material such as acrylic, silicone, latex rubber, or synthetic rubber for example, or other suitable biocompatible material.

The reservoir may be made from a material which is substantially translucent. Alternatively, the reservoir may be made from a material which is substantially the colour of a tooth. For apparatuses to be used with younger persons, it may be desirable to offer the reservoirs and/or mounting arrangements in a number of different colours, or printed with one or more designs.

Generally, the mounting arrangement 11 will be made from a suitable material such as surgical stainless steel, titanium, titanium alloy, or plastic for example, or other suitable biocompatible material.

In use, a desired number of preferred embodiment apparatuses 5 will be fed onto the orthodontic arch wire 3, and the orthodontic wire 3 will be inserted into the channels 1d of the brackets 1. The bands or retainers 5 will retain the arch wire in position in the channels 1d. Due to the configuration of each mounting arrangement 11, the reservoirs will be biased into contact with surfaces of the respective teeth as the orthodontic wire 3 is inserted into the grooves of the brackets 1.

Once the orthodontic wire 3 has been mounted to the brackets 1, the preferred embodiment apparatus will be in a position shown in solid lines in FIG. 2. The reservoir 9 of the preferred embodiment apparatus 5 could be pre-filled commercially with a substance for application to the tooth surface. Alternatively, the reservoir could be filled once the preferred embodiment apparatus has been mounted in the oral cavity. In the embodiment shown, to achieve that the reservoir 9 of the preferred embodiment apparatus is pulled away from the tooth surface against the spring bias, to the position shown in phantom lines in FIG. 2. The reservoir can then be filled with a suitable substance via a bottle 13 or other means. The substance could be provided in liquid, gel, or tablet form for example. Once the substance has been inserted into the reservoir, the reservoir is moved back under the spring bias of the mounting arrangement 11 to the position shown in solid lines in FIG. 2 to bring the reservoir back into contact with the tooth surface for localised application of the substance to the tooth surface.

The preferred embodiment apparatus has the benefit that the reservoir can be moved away from the tooth and refilled, and returned under the spring bias (due to the resilient nature of the arm portions 11b and/or spring bias from the connecting portions 11c) to re-contact the tooth to substantially form a seal and reduce any effect of saliva on the substance to be applied. However, such a configuration is not essential, and there could be provided a relatively rigid mounting means with maintains the reservoir in position against the tooth for localised application of the substance to the surface of the tooth. In order to refill such an embodiment, however, it would be necessary to remove the orthodontic wire 3 from the bracket 1, so it is preferred that a biased configuration is used.

The preferred embodiment apparatus can be used for application of any substance which is suitable for localised application to a tooth surface or other site in the oral cavity, such as fluoride, chlorohexidine, casein phosphopeptide-amorphous calcium phosphate (CCP-ACP)—such as that marketed as a “tooth mousse”, medicaments, whitening agents, or any other suitable substances. The reservoir can hold the substance in any suitable form, such as a liquid, gel, mousse, or solid (such as a tablet) for example. The reservoir could also be used to hold an item that is soaked in the substance to be applied or that acts as a carrier for the substance to be applied.

In the embodiment shown, the (vertical) position of the reservoir relative to the tooth may be altered by adjusting the number of windings of the connecting portion(s) 11c around the arch wire 3. That can be achieved either prior to mounting the preferred embodiment apparatus 7 on the arch wire, or after mounting the preferred embodiment apparatus on the arch wire but before mounting the arch wire to the brackets.

In the embodiment shown, the arch wire has a non-round cross section, to minimise any rotation of the preferred embodiment apparatus about the wire. That assists in maintaining the apparatus in a desired position.

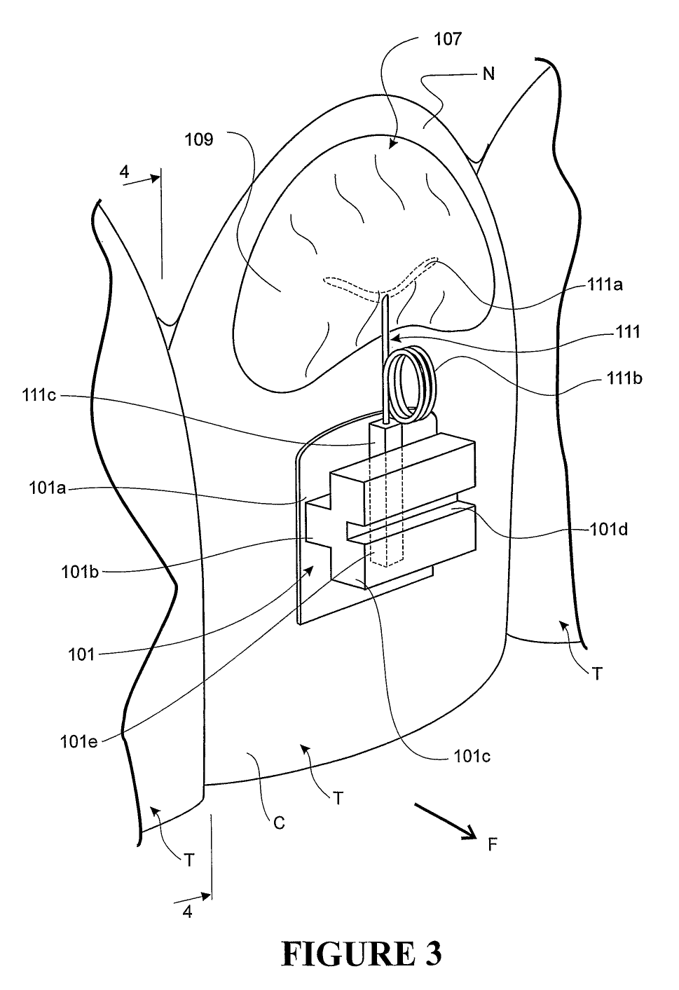

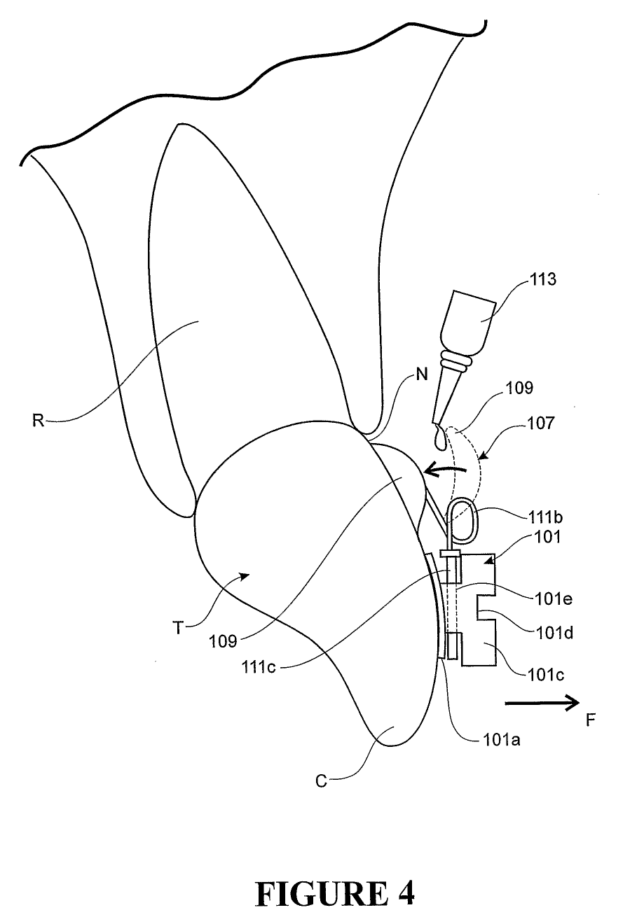

A second preferred embodiment apparatus 107 is shown in FIGS. 3 and 4. Unless described below, the features and operation of this embodiment should be considered the same as for the first embodiment described above and shown in FIGS. 1 and 2, and like reference numerals are used to indicate like parts with the addition of 100.

Again this embodiment has a reservoir 109 for receipt of a substance for localised application to the surface of a tooth T. The reservoir can be considered to be generally the same as in the embodiment of FIGS. 1 and 2. However, the mounting arrangement 111 for this embodiment differs. In this embodiment, the apparatus includes a mounting arrangement 111 which mounts the apparatus to an orthodontic bracket 101 rather than to an orthodontic wire.

Again the orthodontic bracket 101 has base portion 101a which is adhered to a tooth in use, a neck portion 101b, and a body portion 101c which includes a channel 101d for receipt of an orthodontic arch wire. However, in this embodiment the neck portion 101b includes an aperture 101e which is commonly referred to in the art as a vertical slot (shown in phantom lines) for receipt of a portion 111c of the mounting arrangement 111 of the second preferred embodiment apparatus. The mounting arrangement 111 also includes a spring portion 111b which interconnects a portion 111a for engaging the reservoir and the portion 111c. In this embodiment, the connecting portion 111c is a vertical stem which is configured for receipt in the aperture 101e in the neck 101b of the bracket 101. It is preferred that the stem 111c and the aperture 101e are non-circular in cross section, to minimise rotation of the apparatus in the aperture 101e. However, the stem 111c and aperture 101e may be circular and configured for a press-fit engagement.

To mount the second embodiment apparatus within the oral cavity, the stem 111c is pushed into the aperture 101e in the bracket to the desired depth, so that a reservoir 109 is in a desired (vertical) position relative to the tooth. The spring portion 111b will engage the reservoir against the surface of the tooth, as shown in solid lines in FIG. 4. The embodiment shown comprises a torsion spring which supplies a relatively light pressure against the reservoir, sufficient to maintain the reservoir in contact with the tooth but preferably not sufficient to apply any significant torquing action against the tooth.

The apparatus can be pulled outwardly against the spring bias to the position shown in phantom lines in FIG. 4, so that the reservoir can be filled or refilled. Once it has been filled, the reservoir can be returned under the spring bias to re-contact the surface of the tooth.

Instead of having a torsion spring as shown, the mounting arrangement could instead comprise a leaf spring or other mechanism to bias the reservoir into contact with the tooth.

The bracket 101 shown has a channel 101d for receipt of an orthodontic wire 109. That is particularly beneficial as the same bracket can be used for straightening the teeth and supporting the second preferred embodiment apparatus. However, an alternative bracket could be used in embodiments in which it is not desirable to additionally straighten the teeth. Such a bracket could be formed as an integral part of the preferred embodiment apparatus.

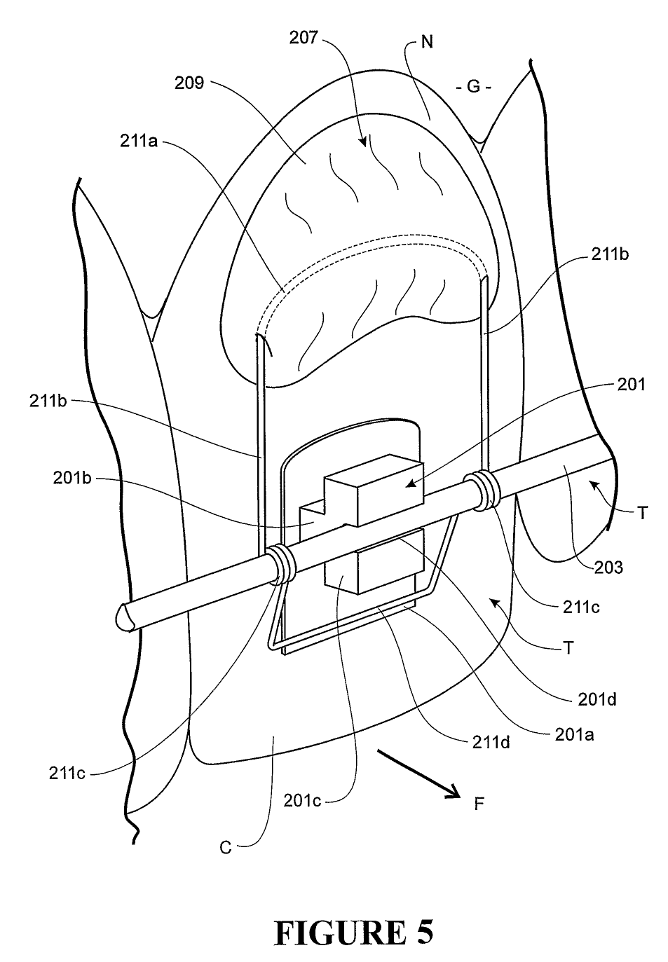

A third preferred embodiment apparatus 207 is shown in FIG. 5. Unless described below, the features and operation of this embodiment should be considered the same as for the first embodiment described above and shown in FIGS. 1 and 2, and like reference numerals are used to indicate like parts with the addition of 200.

In this embodiment, the mounting arrangement 211 differs in that it is configured for use with round cross-section orthodontic arch wire 203 rather than a non-round orthodontic arch wire 3 such as shown in FIGS. 1 and 2. Due to the round orthodontic wire, it can be difficult to get useful biasing of the reservoir about the wire. Accordingly, a portion 211d of the mounting arrangement 211 extends beyond the connecting portions 211c. That portion 211d engages against the base portion 201a of the bracket, and both the reservoir 209 and the portion 211d of the mounting arrangement are biased in the direction of the tooth by the connecting portions 211c.

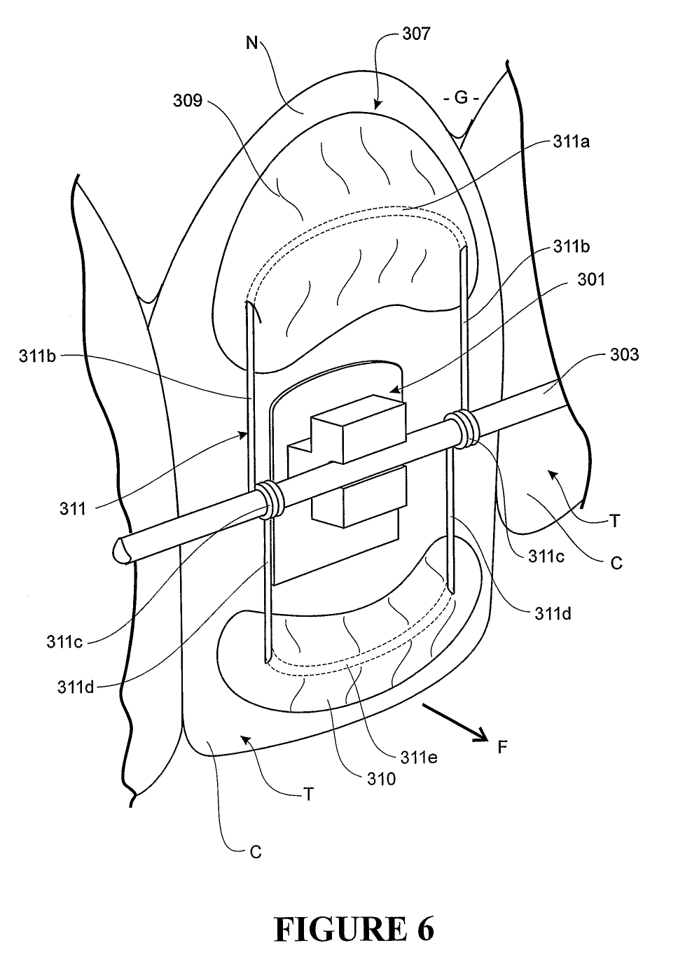

A fourth preferred embodiment apparatus 307 is shown in FIG. 6. Unless described below, the features and operation of this embodiment should be considered the same as for the first embodiment described above and shown in FIGS. 1 and 2, and like reference numerals are used to indicate like parts with the addition of 300.

This preferred embodiment apparatus 301 is a double-acting system, which is suitable for use on a round cross-section orthodontic arch wire 303, although it could also be used on a square or other non-circular orthodontic wire. This apparatus has two reservoirs 309, 310 which are mounted to the arch wire 303 by a mounting arrangement 311. The mounting arrangement includes opposing portions 311a and 311e for engaging respective reservoirs, and which may be moulded into the reservoirs for example. The portions 311a, 311e are mounted on respective arm portions 311b, 311d, which are connected by connecting portions 311c. The connecting portions also extend around the orthodontic wire 303, and preferably act as torsion springs to bias the reservoirs into engagement with the tooth surface.

Prior to mounting the orthodontic wire 303 to the bracket 301 and/or prior to feeding the fourth embodiment apparatus 307 onto the orthodontic wire 303, the number of turns in the connecting portion 311c can be adjusted to alter the positioning of the reservoirs. Once installed, the substance(s) in the reservoirs can be locally applied to two different sites on the tooth, one closer to the gum line and one closer to the exposed tip of the tooth. It is expected such a configuration would only be used in circumstances where it is needed, as it would reduce the useful biting area of the tooth towards the tip of the tooth.

It is envisaged that a kit may be provided comprising a number of preferred embodiment apparatuses of one or more of the forms shown above. That kit could contain a sufficient number to apparatuses to allow application and cosmetic enhancement to a user's upper anterior “smile” teeth, which are the teeth that are readily visible when a person performs an open mouth smile. Most preferably, the kit will contain six apparatuses for the upper six anterior smile teeth, although the kit could contain more or less apparatuses as desired. One or more substances for application by the apparatuses could also be provided in the kit. A number of brackets could be provided in the kit to enable the apparatuses to be mounted to the teeth. The brackets could be an integral part of the apparatuses.

The preferred embodiments of the present invention provide apparatuses which enable reliable localised application of a substance to a site within the oral cavity. The apparatuses are easy to refill with substance if required. The apparatuses are particularly useful for use during procedures with fixed oral appliances such as braces, as they may be used to successfully treat areas of enamel demineralisation prior to permanent discolouration or damage occurring.

While the apparatuses will generally be installed by a dental professional, everyday use can be achieved without professional intervention. Tests with a preferred embodiment apparatus have shown that they can successfully retain a substance such as tooth mousse for at least three weeks. If it is necessary to replace or change the substance in the apparatus, that can be achieved by the end user without expert intervention. Some embodiments, such as those that attach directly to the tooth or have clip portions to enable easy engagement to an orthodontic wire, could also be installed by the user.

The apparatuses can be used for applying single substances, mixtures of substances, or for carrying out a substance application plan which requires the application of one or more substances to the site to be followed up with the application of one or more different substances to the site. For example, once a substance in the reservoir has been expended, more of the same substance or a different substance could be applied. Alternatively, after a substance has been applied for a predetermined time period, more of the same substance or a different substance could then be applied.

The above describes preferred embodiments of the present invention, and modifications may be made thereto without departing from the scope of the invention.

For example, while the embodiments in FIGS. 1 to 5 are shown as positioning the reservoir in the region of the neck of the tooth towards the gum line, the apparatuses could be mounted so that the reservoir is positioned towards the tip of the tooth. Further, while the preferred embodiments are shown as being used on the buccal surfaces of top teeth, they could be used on the buccal surfaces of lower teeth or on the lingual surfaces (facing the tongue) of top or bottom teeth. For use from the lingual surfaces, it will be necessary to mount the preferred embodiment apparatuses on brackets on the lingual surfaces of the teeth.

As another example, other mounting arrangements could be used for mounting the apparatuses in the oral cavity. For example, for embodiments which connect onto an arch wire, the connecting portions 11c could be provided as clips or the like so the apparatuses could be easily removed by an orthodontist or dentist, without removing the arch wire from the brackets.

A number of the shown mounting arrangements have two arms, such as indicated by numeral 11b in FIG. 1. Instead, the mounting arrangement could have a single arm that connects to the reservoir from one side or in an offset configuration.

In some embodiments, it may be desirable that the reservoir is pivotable relative to the arms or engagement portion of the mounting arrangement, to enable the reservoir to be positioned with a suitable angle to accommodate different angled tooth surfaces. That will enhance the contact of the reservoir with the surface to which the substance is to be applied.

The preferred embodiments are described as being used for local application of substances to the surfaces of teeth. However, the apparatuses could be used for local application of a substance or substances to other sites within the oral cavity. For example, the mounting arrangement could be sized and configured such that the reservoir is in contact with a region of the gum which requires treatment due to injury or disease for example. In that embodiment, the apparatus could again be mounted on a bracket on the tooth or an orthodontic arch wire or similar.

The preferred embodiments are described with reference to the human oral cavity, however the apparatuses could be used to locally apply substances in oral cavities of various animal types.

1. An apparatus for localised application of a substance to a site within the oral cavity, comprising a reservoir for receipt of the substance to be applied, and a mounting arrangement for mounting the apparatus within the oral cavity, with the mounting arrangement configured to place the reservoir in contact with the site to which the substance is to be applied to substantially form a seal between the reservoir and the site to which the substance is to be applied.

2. An apparatus as claimed in claim 1, wherein the mounting arrangement is substantially rigid.

3. An apparatus as claimed in claim 1, wherein the mounting arrangement is configured to bias the reservoir into contact with the site to which the substance is to be applied.

4. An apparatus as claimed in claim 3, wherein at least a part of the mounting arrangement is resilient, such that once the apparatus is mounted in the oral cavity, the reservoir may be pulled away from the site against the bias to enable the reservoir to be refilled.

5. An apparatus as claimed in claim 3, wherein the mounting arrangement comprises a spring portion, such that once the apparatus is mounted in the oral cavity, the reservoir may be pulled away from the site against the bias to enable the reservoir to be refilled.

6. An apparatus as claimed in any one of the preceding claims, wherein the mounting arrangement is configured for attachment to an orthodontic appliance.

7. An apparatus as claimed in claim 6, wherein the mounting arrangement is configured for attachment to an orthodontic bracket.

8. An apparatus as claimed in claim 6, wherein the mounting arrangement is configured for attachment to an orthodontic arch wire.

9. An apparatus as claimed in any one of claims 1 to 5, wherein the mounting arrangement comprises a portion that is configured for attachment to a natural part of the oral cavity.

10. An apparatus as claimed in claim 9, wherein the mounting arrangement is configured for attachment to a surface of a tooth.

11. An apparatus as claimed in any one of the preceding claims, wherein the reservoir is pivotable relative to the mounting arrangement, to enhance the contact of the reservoir with the site in the oral cavity to which the substance is to be applied.

12. An apparatus as claimed in any one of the preceding claims, wherein at least a perimeter of the reservoir is relatively flexible, to enhance contact between the reservoir and the site in the oral cavity.

13. An apparatus as claimed in claim 12, wherein the perimeter of the reservoir has greater flexibility than at least a substantial part of the remainder of the reservoir, to enable the substantial part of the remainder to substantially retain its shape for containing the substance to be applied.

14. An apparatus as claimed in any one of claims 1 to 14, wherein the apparatus is configured to apply the substance to a surface of a tooth in the oral cavity.

15. An apparatus as claimed in claim 14, wherein the apparatus is configured to apply the substance to a neck region of a tooth in the oral cavity.

16. An apparatus as claimed in claim 15, wherein the apparatus is configured to apply the substance to a neck region of a buccal surface of a tooth in the oral cavity.

17. An apparatus as claimed in any one of the preceding claims, comprising more than one reservoir, such that the apparatus is capable of concurrently locally applying one or more substances to more than one site in the oral cavity.

18. A kit comprising a plurality of apparatuses as claimed in any one of the preceding claims.

19. A kit as claimed in claim 18, comprising six of said apparatuses.

20. A kit as claimed in claim 18 or 19, further comprising at least one substance for application by the apparatuses.

21. A method of locally applying a substance to a site within an oral cavity, comprising mounting an apparatus as claimed in any one of claims 1 to 17 in the oral cavity; and applying a substance from the reservoir to the site in the oral cavity.

22. A method as claimed in claim 21, wherein the apparatus has been pre-filled with the substance.

23. A method as claimed in claim 21, comprising inserting the substance into the reservoir after mounting the apparatus in the oral cavity.

24. A method as claimed in any one of claims 21 to 23, wherein the method comprises, once a substance has been expended or following a predetermined time delay, moving the reservoir out of contact with the site, inserting more substance or a different substance in the reservoir, and bringing the reservoir back into contact with the site.

25. A method as claimed in any one of claims 21 to 24, when used to cosmetically enhance a user's teeth.
